(19) United States
(12) Patent Application Publication
(10) Pub. No.: US 2026/0335566 A1
(43) Pub. Date: Oct. 29, 2026

(54) CARTRIDGE FOR INHALATION DEVICE

(71) Applicant: invoX Belgium NV, Diepenbeek (BE)

(72) Inventors: Jürgen Rawert, Cologne (DE); Steven Madden, Hamburg (DE)

(21) Appl. No.: 19/492,147

(22) PCT Filed: Jun. 27, 2024
(86) PCT No.: PCT/EP2024/068149

§ 371 Date: Dec. 11, 2025

(30) Foreign Application Priority Data
Jun. 30, 2023 (EP) 23182849.2

(51) Int. Cl. A61M 11/00; A61M 11/08

(52) U.S. Cl. CPC A61M 11/007; A61M 11/08

(57) ABSTRACT

The invention provides a cartridge (1) for an inhalation device for the generation and administration of a medically active liquid (2) in aerosolized form, the cartridge comprising—a dimensionally stable outer casing (10), and—a collapsible inner tube (20) for holding the medically active liquid, being located inside the outer casing in the assembled state of the cartridge, wherein the collapsible inner tube comprises an outlet end (21) having an outlet opening (22) and a closed end (23) opposite of the outlet end, the outlet opening and the (the center of the) closed end defining a main axis (X) of the inner tube connecting the outlet opening and closed end of the tube, wherein the closed end of the tube being closed by a welded seam (24) oriented transverse (preferably substantially perpendicular) to the main axis of the inner tube; and wherein the outer casing comprises a deformation element (11) contacting the welded seam (in the assembled state of the cartridge) such that the welded seam is deformed to a radially curved form by the contact between the welded seam and the deformation element.

### FIELD OF THE INVENTION

The present invention relates to the field of inhalation devices for medically active liquids. In particular, the invention relates to a cartridge for an inhalation device for the generation and administration of a medically active liquid (2) in aerosolized form, the cartridge comprising a dimensionally stable outer casing and a collapsible inner tube. Furthermore, the present invention relates to an inhalation device comprising such cartridge as well as a method for the assembly of such cartridge.

### BACKGROUND OF THE INVENTION

Nebulizers or other aerosol generators for liquids have long been known from the art. Amongst others, such devices are used in medical science and therapy. There, they serve as inhalation devices for the application of active ingredients in the form of aerosols, i.e., small liquid droplets embedded in a gas. Such an inhalation device is known, e.g., from document EP 0 627 230 B1. Essential components of this inhalation device are a reservoir, often in the form of a cartridge, in which the liquid that is to be aerosolized is contained; a pumping device for generation of a pressure being sufficiently high for nebulizing the liquid; as well as an atomizing device in the form of a nozzle. By means of the pumping device, the liquid is drawn in a discrete amount, i.e., not continuously, from the reservoir and fed to the nozzle. The pumping device works without propellant and generates pressure mechanically.

Cartridges for holding the medically active liquids are, for example, described in US 2004/0143235 A1. The document discloses a container for a medicinal liquid, the container being gas-tight and liquid-tight so that it is storable over many months. The container includes a collapsible foil bag on which a flange, which is stable in respect of shape, is disposed. The flange is designed for fitting onto a discharge connection member. The container can be disposed in a casing which is stable in respect of shape. The medicinal liquid does not come into contact with air and is protected from the effect of light. The medicinal liquid may be dispensed from the foil bag in many partial quantities over a prolonged period of time, with a respective partial amount thereof being converted into an aerosol by means of an atomizer.

WO 2009/115200 A1 discloses a reservoir for a nebulizer, a nebulizer and a method of filling a reservoir. To avoid undesirable rises in pressure a fluid chamber of the reservoir is pre-collapsed and filled with an initial amount of fluid which is less than the maximum volume of the fluid chamber. A device may be used to pre-collapse the fluid chamber, wherein the device may have at least manipulating or abutment element. As a result of the external placement of the abutment elements against the wall of the fluid chamber or the deformation thereof, the actual volume of the fluid chamber available during the filling with the fluid is reduced, i.e., it is less than the maximum volume of the fluid chamber. This results in the desired pre-collapsing.

A technical problem associated with the withdrawal of a the medically active fluid from such fluid bags or chambers, especially when withdrawn in a plurality of discrete small amounts, however, is the formation of an unwanted underpressure inside the foil bag or chamber which can potentially affect the precise dosing of the medically active liquid as well as the withdrawal of the intended total amount of medically active liquid to be provided by the fluid bag or chamber.

It is thus an object of the present invention to provide for a cartridge for an inhalation device that allows for the storage of a broad variety of medically active liquids including aqueous or organic liquids and for the withdrawal or dispensing thereof, especially in a plurality of discrete steps. A further object of the present invention is to provide a cartridge for an inhalation device that allows for the withdrawal or dispensing of a medically active liquid in a large number of precise consecutive aliquots or doses. Further objects of the invention will be clear on the basis of the following description of the invention, examples and claims.

### SUMMARY OF THE INVENTION

In a first aspect, the invention relates to a cartridge (1) for an inhalation device for the generation and administration of a medically active liquid (2) in aerosolized form, the cartridge comprising
a dimensionally stable outer casing (10),
at least one deformation element (11), and
a collapsible inner tube (20) for holding the medically active liquid comprising:
   an outlet end (21) having an outlet opening (22) and
   a closed end (23) opposite of the outlet end, wherein the outlet opening and the (the center of the) closed end defining a main axis (X) of the inner tube connecting the outlet opening and closed end of the tube;
   wherein the closed end of the tube is closed by a welded seam (24) oriented transverse (preferably substantially perpendicular) to the main axis of the inner tube;
wherein in the assembled state of the cartridge the collapsible inner tube is located inside the outer casing;
characterized in that
in the assembled state the deformation element (11) contacts the welded seam such that the welded seam is deformed to deviate from a traversal line to the main axis of the inner tube by the contact between the welded seam and the deformation element.

In a second aspect the invention relates to an inhalation device adapted to utilize a cartridge according to the first aspect of the invention.

In a third aspect the invention relates to an inhalation system comprising an inhalation device (100) adapted to comprise a cartridge according to invention and a cartridge according to the invention.

In a fourth aspect the invention relates to a method for the assembly of a cartridge according to the invention, the method comprising the steps of
a) providing an inner tube and a dimensionally stable outer casing comprising at least one deformation element;
b) introducing the inner tube into the outer casing, thereby contacting the welded seam of the inner tube with the at least one deformation element and deforming the welded seam of the inner tube;
c) filling of the inner tube with the medically active liquid;
d) optionally sealing the cartridge after filling.

### DESCRIPTION OF THE DRAWINGS

FIG. 1 depicts a schematic of a cartridge for an inhalation device.

FIGS. 2A, 2B and 2C depicts a cross-section of a cartridge for an inhalation device according to the present invention. FIG. 2A shows the cartridge in an assembled state. FIGS. 2B and 2C show cross-sections of the parts of the cartridge.

FIGS. 3A, 3B and 3C illustrate the deformation of the welded seam of the inner tube.

FIG. 4 shows an exemplified inhaler with a cartridge.

### DETAILED DESCRIPTION OF THE INVENTION

The inventors found that the disadvantages of the prior art can be overcome with a new cartridge design, which is particularly suitable for cartridges comprising an inner tube made from stable and/or rigid materials, e.g., thicker plastic or metal. It was found by the inventors that the dosing and removal of fluid from an inner tube comprising said fluid can be improved if said inner tube comprises a welding seam and said welding seam is deformed to deviate from an ideal straight line.

Accordingly, in a first embodiment, the invention relates to a cartridge (1) for an inhalation device for the generation and administration of a medically active liquid (2) in aerosolized form, the cartridge comprising
a dimensionally stable outer casing (10),
at least one deformation element (11), and
a collapsible inner tube (20) for holding the medically active liquid, the collapsible inner tube comprising:
   an outlet end (21) having an outlet opening (22) and
   a closed end (23) opposite of the outlet end, wherein the outlet opening and the (the center of the) closed end defining a main axis (X) of the inner tube connecting the outlet opening and closed end of the tube;
   wherein the closed end of the tube is closed by a welded seam (24) oriented transverse (preferably substantially perpendicular) to the main axis of the inner tube;
wherein in the assembled state of the cartridge the collapsible inner tube is located inside the outer casing;
characterized in that
   in the assembled state the deformation element (11) contacts the welded seam such that the welded seam is deformed to deviate from an ideal traversal line to the main axis of the inner tube by the contact between the welded seam and the deformation element.

In the context of the present invention the term “welded seam” refers to a welded seam present opposite of the outlet end of the inner tube. The inner collapsible tube may comprise additional welded seams, but in preferred embodiments, the inner collapsible tube comprises a single welded seam closing one end of the tube.

In some embodiments, the welded seam extends transversely with respect to the main axis (X) of the bag. Preferably, the welded seam is of a rectangular shape, however, the welded seam may be in other shapes, such as a U-shape. In a preferred embodiment, the welded seam is of rectangular shape.

The welded seam preferably has a welded height sufficient to effectively close the bag, and allows for a deformation of said seam, without substantially causing a precompression in the non-welded part of the inner collapsible tube.

The welded seam preferably has a height of at least 0.1 mm, and up to 5 mm. In some embodiments, the height is between 0.1 to 5 mm, preferably between 0.1 and 2 mm, more preferably between 0.3 to 6 mm. In preferred embodiments, the seam is as small as possible, preferably 3 mm height or smaller, more preferably between 0.5 mm to 2 mm. In some embodiments, the seam has a height selected from 0.1 mm, 0.2 mm, 0.3 mm, 0.4 mm, 0.5 mm, 0.75 mm, 1 mm, 1.25 mm, 1.5 mm, 1.75 mm, and 2 mm.

The collapsible inner tube is made from any suitable material. In particular, suitable materials include materials that allow for the generation of a collapsible tube, which allows however, for a still rigid kind of tube. Preferred materials show a stable form and are not substantially deformed when the tube collapses due to the removal of fluid. Preferably, the collapsible inner tube maintains the general overall shape and essentially constricts on the removal of fluid.

The material used for the collapsible inner tube is also dependent on the fluid to be used. In some embodiments of the invention the cartridge is for an ethanol-based fluid. Accordingly, in some embodiments, the collapsible inner bag is made of or comprises a material impermeable for ethanol. In preferred embodiments, the collapsible inner tube is impermeable for ethanol.

Suitable materials include polyethylene, in particular high-density polyethylene, nylon, ethylene vinyl alcohol (EVOH), polyamide and polypropylene, or metals such as aluminum or mixtures of different materials.

In some particular embodiments, the collapsible inner tube is made of multiple materials. In some embodiments, the tube is made or comprises layers of different materials. If the tube comprises different materials, the tube may be adapted according to the requirements of the fluid. One layer of material may provide sufficient stability of the inner tube, and a second material layer may provide impermeability for the fluid.

In a particular embodiment, the invention relates to a cartridge as defined above-wherein the inner collapsible bag comprises at least two distinct material layers. In some embodiments, the inner collapsible tube comprises three or more layers.

In a particular embodiment of the invention, the collapsible inner bag comprises at least three layers: an outer layer comprising a polymer and/or co-polymer; a central layer comprising a metal layer and an inner layer comprising a polymer and/or or co-polymer. In the context of the invention, the term outer layer, refers to the layer facing the outside of the collapsible tube and the inner layer refers to the layer facing the inside of the collapsible tube. The term central layer refers to a layer positioned between the inner and outer layer.

The collapsible inner tube may be of any suitable general shape. In some embodiment, the collapsible tube is essentially tube-shaped. In particular, the collapsible tube comprises aside from the welded seam a pouch portion and a collar portion. In these embodiments, the pouch portion is generally closed at one end by the welded seam and connects into the collar portion. The collar portion preferably comprises the outlet opening. In some embodiments, the collar portion is further adapted to be attached or connected to the outer shell of the casing.

Accordingly, in some embodiments the invention relates to a cartridge as defined above, wherein the collapsible tube comprises a pouch portion (26) and a collar portion (27), wherein the collar portion comprises the outlet opening and is adapted to be attached to the shell of the outer casing of the cartridge.

In some embodiments, the invention relates to a cartridge as defined above, wherein the pouch portion of the collapsible tube comprises polyethylene, in particular high-density polyethylene, nylon, ethylene vinyl alcohol (EVOH), polyamide and polypropylene, or metals such as aluminum or mixtures thereof. In some embodiments, the pouch portion comprises multiple material layers, wherein each layer independently comprises polyethylene, in particular high-density polyethylene, nylon, ethylene vinyl alcohol (EVOH), polyamide and polypropylene, or metals such as aluminum or mixtures thereof.

In some embodiments, the invention relates to a cartridge as defined above, wherein the collar portion of the collapsible tube comprises polyethylene, high density polyethylene, polylactic acid, and/or polyoxymethylene. The selected material should be compatible with the material of the pouch portion.

The selection of the material also allows for the selection of the thickness of the walls of the collapsible inner bag, in particular of a pouch portion (26) of the collapsible inner bag. The thickness may be adjusted depending on the requirements of the fluid and inhaler type. It is preferred that the walls of the collapsible inner tube, aside from the welded seam have a thickness of 1 mm or less. In some embodiments, the wall of the inner tube has a thickness between 100 and 250 μm. In some embodiments, the thickness is between 125 and 225 μm. In preferred embodiments the thickness is between about 150 and 200 μm. In a particular embodiment, the thickness is about 175 μm.

The inventors found that the withdrawal of fluid from an inner tube with a welded seam is improved and better controllable if said welded seam is deformed in the cartridge. In the context of the present invention the term deformed to deviate from an ideal traversal line means that the welded seam does not follow a straight line, corresponding to a 180° angle, but deviates from an 180° angle. The deviation may be that the welded seam is curved, angled, bent, warped or otherwise distorted, preferably radially distorted.

The deviation can be defined by the deviation from the ideal traverse line to the main axis of the collapsible inner tube. The furthest point of the welded seam may ideally deviate at least 1 mm, preferably 1.5, more preferably 2 mm from the ideal straight traverse line. In some embodiments the welded seam may deviate up to 0.5 mm, 1 mm, 1.5 mm, 2 mm, 2.5 mm, 3 mm, 3.5 mm, 4 mm, 4.5 mm, or 5 mm from an ideal straight line at the furthest point.

Said deviation is generated by the deformation element. In the contest of the present invention, the term “deformation element” refers to any element of the cartridge that interacts or contacts the welded seam of the collapsible inner tube and causes the deformation of the welded seam.

Said deformation of the welded seam does not necessarily contribute to a pre-collapsing of the collapsible inner tube. The invention is particularly suitable for collapsible inner tubes made from more rigid materials. The deformation of the welded seam allows for controlled collapsing of said tube. In contrast to the reservoir described in WO 2009/115200 A1, the problem to be solved is not an undesired increase in pressure inside the inner collapsible tube, but an avoidance of an underpressure as the tube might not fully collapse when fluid is extracted. This problem can be avoided with the deformation of the welded seam.

The deformation element is preferably part of the dimensionally stable outer casting. Said outer casting may be a single part or comprise multiple parts. In some embodiments, the dimensionally stable outer casing is a single piece, comprising an opening which allows the insertion of the collapsible inner tube and said deformation element. In some embodiments, the dimensionally stable outer casing comprises multiple components. For example, the dimensionally stable outer casing may comprise multiple parts, such as a casing as defined above and a cap for securing the collapsible inner tube.

In a preferred embodiment, the invention relates to a cartridge as defined above, wherein the outer casing comprises or consists of
a shell (12) comprising an outlet opening and an opposite base opening;
a cap (13) adapted to be attached to the outlet opening of the shell; and
a base (14) adapted to be attached to the base opening of the shell.

The outer casing may comprise additional components, such as a cartridge extension as described in WO 2011/198151 A1. Said cartridge extension may interact with other part of the inhaler, e.g., in the form of a combined counting and blocking device as described in WO 2023/001750 A1.

The deformation element is positioned in the dimensionally stable outer casing in a way that it is possible to deform the welded seam as defined above. As such, in most embodiments the deformation element is present opposite of the opening of the casing. In embodiments, in which dimensionally stable outer casing comprises a bases, it is preferred that the deformation element is provided on the base of the outer casing.

Accordingly, in a preferred embodiment, the invention relates to a cartridge, wherein the outer casing comprises
a shell (12) comprising an outlet opening and an opposite base opening;
a cap (13) adapted to be attached to the outlet opening of the shell; and
a base (14) adapted to be attached to the base opening of the shell, and
   wherein the deformation element is provided on the base of the outer casing.

The outer casing and its parts may be made of any suitable material. In some embodiments, the outer casing and its parts are made of the same or partially the same materials as the collapsible inner tube. If the outer casing is made of multiple parts, including but not limited to a shell a cap and a base, these parts may be made of the same or different materials.

In some embodiments, the invention relates to a cartridge as defined above, wherein the shell and/or base of the outer casing comprises high density polyethylene, polyethylene, polypropylene, polyoxymethylene, metal, e.g., aluminum, acrylonitrile butadiene styrene, or combinations and mixtures thereof.

In some embodiments, the invention relates to a cartridge as defined above, wherein the cap of the outer casing comprises low density polyethylene, polyethylene, an elastomer, silicones, ethylene propylene diene monomer rubber, metal/elastomer combinations and or combinations and mixtures thereof.

An advantage of the present invention is that the volume of the cartridge can be adapted as needed for the requirements of the nebulizer. The volume of the outer casing can be adjusted to accommodate the size of the collapsible inner tube. As long as the volume of the outer casing is sufficient to include the collapsible inner tube, the outer casing may have any volume required by the nebulizer.

In some embodiments of the invention, the outer casing has a volume in the range of between about 0.1 to 200 ml. In particular embodiments the volume of the outer casing is between about 0.5 to about 50 ml. In particular embodiments, the volume of the outer casing is between about 1 mL to 10 ml.

The volume of the collapsible inner bag is generally smaller than the volume of the casing so that the collapsible inner tube is able to fit within the outer casing. In particular it is preferred that the volume of the collapsible inner tube is between 0.1 and 0.9 times the volume of the dimensionally stable outer casing. In some embodiments, the volume is about 0.3 to 0.7 times the volume of the outer casing. In some embodiments the volume of the collapsible inner tube is selected from 0.1, 0.2, 0.3, 0.4, 0.5, 0.7, 0.8, and 0.9 times the volume of the outer casing.

In some embodiments of the invention, the inner tube has a volume in the range of from about 2 to about 8 ml, In some embodiments the volume of the inner tube is selected from 2 ml, 2.5 ml, 3 ml, 3.5 ml, 4 ml, 4.5 ml, 5 ml, 5.5 ml, 6 ml, 6.5 ml, 7 ml, 7.5 ml and 8 ml. In some specific embodiments the volume of the inner tube is about 3 ml.

The deformation element may be provided in any suitable form. Preferably, the deformation element is provided in the form of a protrusion or multiple protrusions. Said protrusions may be of any suitable shape. In some embodiments said protrusions may be in the form of guiding walls. In a different embodiment, the deformation element may be an indentation, for example in the form of a guiding channel or a groove that receives the welded seam.

If the deformation element is provided in the form of a protrusion, it is preferred that said deformation element is oriented parallel to the main axis (X) of the inner tube. In some specific embodiments at least one deformation element is oriented on the main axis (X) of the inner collapsible tube.

In some embodiments the deformation element is a protrusion or a set of protrusions. In preferred embodiments set protrusion is pin-shaped. Accordingly, in a particular embodiment, the invention relates to a cartridge as defined above, wherein the deformation element is provided in the form of at least one pin (15) extending from the base of the outer casing. Said pin may be of any reasonable shape, however, it is preferred that the pin has a generally round shape. In preferred embodiments, the deformation element is provided in the form of a pin, wherein said pin has a circular cross section.

In preferred embodiments, the deformation element is a protrusion, e.g., a pin or a plurality of pins as defined above, extending from the bottom of the dimensionally stable casing toward the opening of the dimensionally stable casing. In some embodiments of the invention, wherein the at least one deformation element is provided in form of at least one pin, it is preferred that the at least one pin is oriented parallel to the main axis (X) of the inner tube.

In some embodiments of the invention, the deformation element is provided in the form of a plurality of pins. Any suitable number of pins may be used. The number and placement of pins allows to adapt and adjust the deformation as needed, depending on the collapsible inner bag. A particularly suitable arrangement is based on three pins. Accordingly, in one embodiment the invention relates to a cartridge as defined above, wherein the deformation element is provided in the form of three pins. In a particular embodiment, said three pins are located on a base plate, as defined above, such that upon contact and deformation of the welded seam one pin is located on one broadside (25) of the welded seam and two pins are located on the opposite broadside of the welded seam.

As noted before, it is preferable, that the deformation element does not interact with the non-welded parts of the collapsible inner bag. As such, in some embodiments of the invention the deformation element does not substantially contact the collapsible sidewalls of the collapsible tube. In preferred embodiments, the deformation element only contacts the welded seam of the collapsible inner tube. In particular, the deformation element is preferably designed to not substantially reduce the inner volume of the collapsible inner tube.

Accordingly, in some embodiments of the invention, the invention relates to a cartridge as defined above, wherein the deformation element upon contact with the welded seam of the collapsible tube does not substantially reduce the inner volume of the collapsible tube.

The cartridge according to the present invention is intended for use in an inhalation device for the administration of a medically active liquid. As such, in one embodiment of the invention, the cartridge, and in particular, the collapsible inner bag comprises a medically active liquid. Said medically active liquid should be administered by inhalation.

The cartridge is suitable for any medically active liquid that is administer by inhalation. In some embodiments this might be a liquid active agent. In other embodiments the medically active liquid comprises a pharmaceutically active ingredient and a liquid vehicle. The medically active liquid may be any kind of suitable liquid.

In particular embodiments, the invention relates to a cartridge as defined above, comprising a medically active liquid, wherein the medically active liquid comprises at least one pharmaceutically active ingredient and a liquid vehicle.

Liquid vehicles suitable for medically active compounds are known to the skilled persons. Such liquid vehicles include solutions, suspensions and emulsions. Preferred liquid vehicles include solvents. In some embodiments the liquid vehicle comprises at least one solvent selected from the group consisting of water, ethanol, polyethylene glycol, liquid fatty acids, oils, in particular natural oils, and semifluorinated alkanes. In a particular preferred embodiment, the liquid vehicle comprises a solvent, wherein the solvent is ethanol.

In a further aspect of the invention, the invention relates to a method for the assembly of a cartridge as defined above, the method comprising the steps of
a) providing a collapsible inner tube and a dimensionally stable outer casing comprising a deformation element;
b) introducing the inner tube into outer casing, thereby contacting the welded seam of the inner tube with the deformation element of the outer casing and deforming the welded seam of the inner tube;
c) filling of the inner tube with a medically active liquid;
d) optionally sealing the container after filling.

In preferred embodiments, step (d) is not optional.

In a further aspect the invention relates to an inhalation device (100) adapted to comprise a cartridge as defined above. In a particular embodiment, the invention relates to a kit comprising an inhalation device (100) adapted to comprise a cartridge as defined above and a cartridge according to any one of the preceding claims. The cartridge may be removably or permanently comprised in the inhalation device.

The inhalation device may be any kind of liquid inhalation device. In preferred embodiments, the inhalation device is a soft mist inhalation device, preferably a soft mist inhalation device comprising an impingement type nozzle.

In a further aspect, the invention relates to a method for the assembly of a cartridge as defined above, the method comprising the steps of
a) providing an inner tube and a dimensionally stable outer casing comprising a deformation element;
b) introducing the inner tube into outer casing, thereby contacting the welded seam of the inner tube with the deformation element of the outer casing and deforming the welded seam of the inner tube;
c) filling of the inner tube with the medically active liquid;
d) optionally sealing the container after filling.

The invention further relates to the following numbered embodiments:
1. A cartridge (1) for an inhalation device for the generation and administration a medically active liquid in aerosolized form, the cartridge comprising
   a dimensionally stable outer casing (10),
   a deformation element (11), and
   a collapsible inner tube (20) for holding the medically active liquid comprising:
      an outlet end (21) having an outlet opening (22) and
      a closed end (23) opposite of the outlet end, wherein the outlet opening and the (the center of the) closed end defining a main axis (X) of the inner tube connecting the outlet opening and closed end of the tube;
      wherein the closed end of the tube is closed by a welded seam (24) oriented transverse (preferably substantially perpendicular) to the main axis of the inner tube;
wherein in the assembled state of the cartridge the collapsible inner tube is located inside the outer casing;
characterized in that
   in the assembled state the deformation element (11) contacts the welded seam such that the welded seam is deformed to deviate from a traversal line (L) to the main axis of the inner tube by the contact between the welded seam and the deformation element.
2. The cartridge according to embodiment 1, wherein the outer casing comprises
   a shell (12) comprising an outlet opening and an opposite base opening;
   a cap (13) adapted to be attached to the outlet opening of the shell; and
   a base (14) adapted to be attached to the base opening of the shell.
3. The cartridge according to embodiment 2, wherein the deformation element is provided on the base of the outer casing.
4. The cartridge according to embodiment 2 or 3, wherein the deformation element is provided in the form of at least one pin (15) (preferably having a circular cross-section) extending from the base of the outer casing (in the direction of the outlet opening).
5. The cartridge according to embodiment 4, wherein the at least one pin is oriented parallel to the main axis (X) of the inner tube.
6. The cartridge according to embodiment 4 or 5, wherein the deformation element is provided in the form of a plurality of pins.
7. The cartridge according to any one of embodiments 4 to 6, wherein the deformation element is provided in the form of three pins.
8. The cartridge according to embodiment 7, wherein the three pins are located on the base plate such that upon contact and deformation of the welded seam one pin is located on one broadside (25) of the welded seam and two pins are located on the opposite broadside of the welded seam.
9. The cartridge according to embodiment 3, wherein the deformation element is provided in the form of groove provided on the base of the outer casing (for receiving the welded seam of the collapsible tube).
10. The cartridge according to any one of the preceding embodiments, wherein the deformation element contacts the welded seam of the collapsible tube only and does not substantially contact the collapsible sidewalls of the collapsible tube.
11. The cartridge according to any one of the preceding embodiments, wherein the deformation element upon contact with the welded seam of the collapsible tube does not substantially reduce the inner volume of the collapsible tube.
12. The cartridge according to any one of the preceding embodiments, wherein the collapsible tube comprises a pouch portion (26) and a collar portion (27), wherein the collar portion comprises the outlet opening and is adapted to be attached to the shell of the outer casing of the cartridge.
13. The cartridge according to embodiment 12, wherein the pouch portion of the collapsible tube comprises polyethylene, in particular high-density polyethylene, nylon, ethylene vinyl alcohol (EVOH), polyamide and polypropylene, or metals such as aluminum or mixtures thereof.
14. The cartridge according to embodiment 12 or 13, wherein the collar portion of the collapsible tube comprises polyethylene, high density polyethylene, polylactic acid, and/or polyoxymethylene.
15. The cartridge according to any one of embodiments 2 to 14, wherein the shell of the outer casing comprises high density polyethylene, polyethylene, polypropylene, polyoxymethylene, metal, e.g., aluminum, acrylonitrile butadiene styrene, or combinations and mixtures thereof.
16. The cartridge according to any one of embodiments 2 to 15, wherein the cap of the outer casing comprises low density polyethylene, polyethylene, an elastomer, silicones, ethylene propylene diene monomer rubber, metal/elastomer combinations and or combinations and mixtures thereof
17. The cartridge according to any one of embodiments 2 to 16, wherein the base of the outer casing comprises high density polyethylene, polyethylene, polypropylene, polyoxymethylene, metal, e.g., aluminum, acrylonitrile butadiene styrene, or combinations and mixtures thereof.
18. The cartridge according to any one of the preceding embodiments, wherein the outer casing has a volume in the range of from about 0.1 to about 200 mL, optionally about 0.5 to about 50 ml, further optionally between about 1 mL to 10 ml.
19. The cartridge according to any one of the preceding embodiments, wherein the inner tube has a volume in the range of from about 2 to about 8 mL.
20. The cartridge according to any one of the preceding embodiments, wherein the radially curved form of the welded seam comprises a deviation of up to 0.5 mm, 1 mm, 1.5 mm, 2 mm, 2.5 mm, 3 mm, 3.5 mm, 4 mm, 4.5 mm, or 5 mm from an ideal straight line at the furthest point.
21. The cartridge according to any one of the preceding embodiments, wherein the medically active liquid comprises at least one pharmaceutically active ingredient and a liquid vehicle.
22. The cartridge according to embodiment 21, wherein the liquid vehicle comprises at least one solvent selected from the group consisting of water, ethanol, polyethylene glycol, liquid fatty acids, oils, in particular natural oils, and semifluorinated alkanes.
23. An inhalation device (adapted to comprise a cartridge according to any of the preceding embodiments) comprising a cartridge according to any one of the preceding embodiments.
24. A method for the assembly of a cartridge according to any one of embodiments 1 to 22, the method comprising the steps of
   a) providing an inner tube and a dimensionally stable outer casing comprising a deformation element;
   b) introducing the inner tube into outer casing, thereby contacting the welded seam of the inner tube with the deformation element of the outer casing and deforming the welded seam of the inner tube;
   c) filling of the inner tube with the medically active liquid;
   d) optionally sealing the container after filling.

### DETAILED DESCRIPTION OF THE DRAWINGS

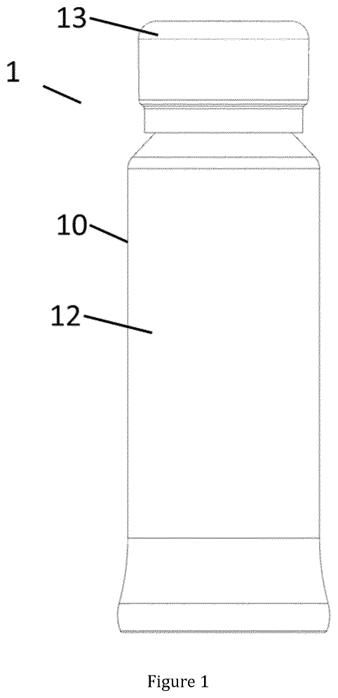

FIG. 1 shows a schematic of a cartridge (1) for an inhalation device (30) in assembled form. The cartridge (1) comprises a dimensionally stable outer casing (10) which comprises at least an outer shell (12) and a cap (13) closing of the cartridge (1). The collapsible inner tube (20) is not visible in this figure.

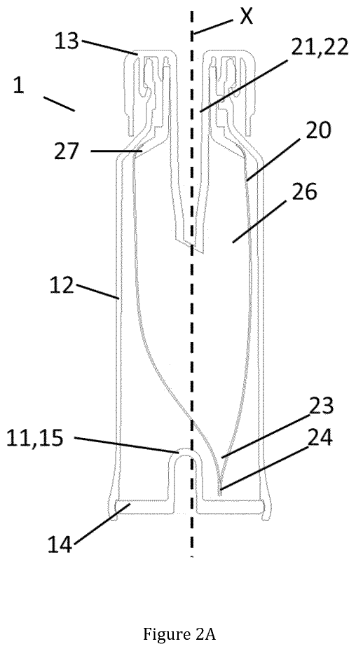

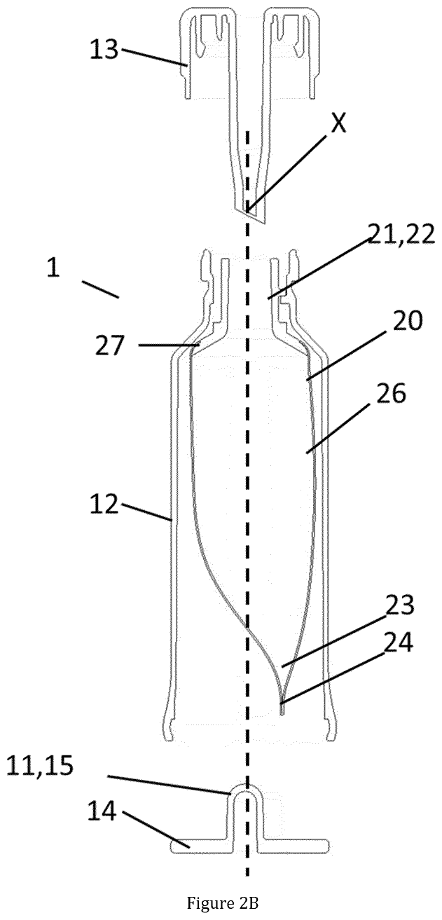

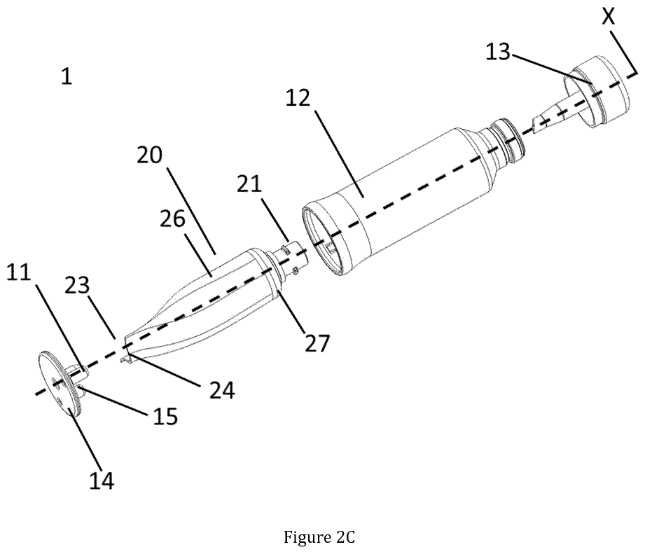

FIGS. 2A and 2B show a cross-section of a cartridge (1) for an inhalation device (30) according to the present invention. FIG. 2A shows the cartridge (1) in assembled state. In the depicted embodiment the dimensionally stable outer casing (10) of the cartridge (1) comprises a base (14), an outer shell (12) and a cap (13). The base comprises a deformation element (11) in the form of a pin (15). The collapsible inner tube (20) is inserted in the dimensionally stable outer casing (10) secured by the cap (13). The collapsible inner tube comprises an outlet end (21) with an outlet opening (22) interacting with the cap (13) of the outer casing. The closed end (23) is closed by a welded seam (24) which is contacted by the deformation element (11) to cause a deviation from a straight line. The main axis (X) of the cartridge is outlined to indicate the deviation of the welded seam. The collapsible inner tube (20) comprises a pouch portion (26) which is filled with the medically active liquid and ends in the welded seam on the one side and a collar portion (27) on the other side. The collar portion marking the outlet end and providing the outlet opening.

FIG. 2B shows the same embodiment of the cartridge (1) in a disassembled state. The Figure in particular depicts a cross-section of the base (14) of the dimensionally stable outer casing (10) comprising the deformation (11) element in form of a pin (15). The figure allows further a better depiction of the collapsible inner tube (20), still inserted in the shell (12) of the outer casing (10). The collapsible inner tube comprises an out let end (21) with outlet opening (22), closed end (23) with welded seam (24) and pouch portion (26).

FIG. 2C shows further depictions the elements of the cartridge in stylized form, not in the form of a cross section. The figure depicts the different components of the cartridge (1) prior to assembly: Base (14) collapsible inner bag (20), shell (12) and cap (13) oriented along the main axis (X) of the cartridge.

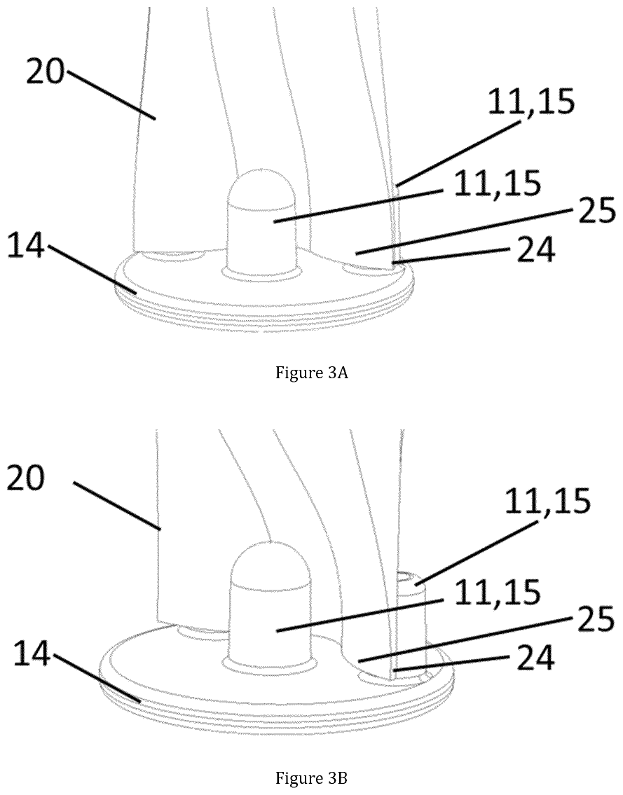

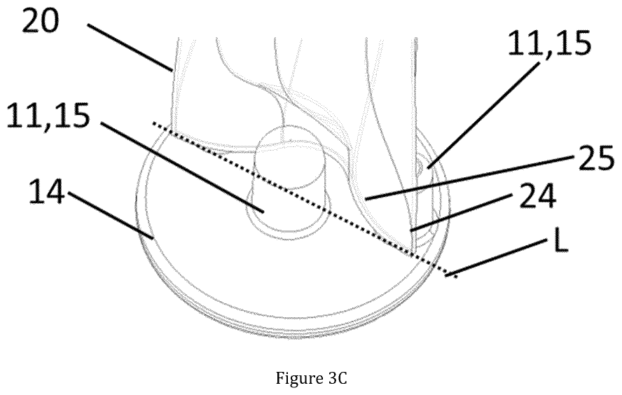

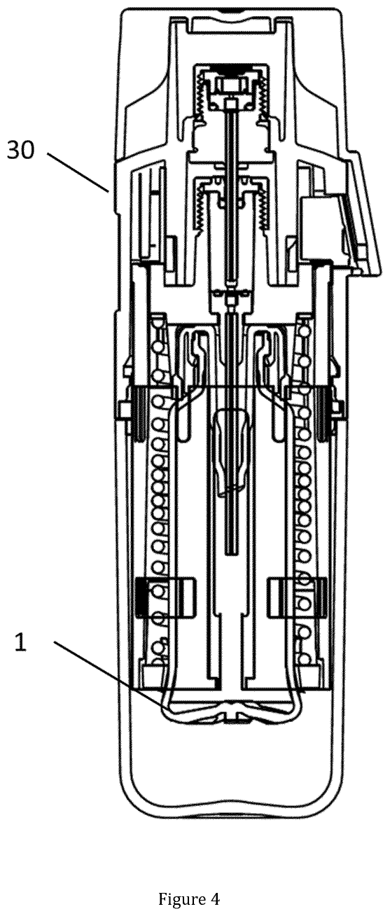

FIGS. 3A, 3B and 3C show different angles of the base (14) with the deformation element (11) in the form of a pin (15) interacting with the broadside (25) of the welded seam (24) of the collapsible inner tube (20). All figures show the deformation of the welded seam (24) to a radially curved form deviating from an ideal traversal line (L) to the main axis (X). FIG. 3C illustrates and depicts the traversal line (L) to show the deviation.

FIG. 4 shows an inhaler (30) with an inserted cartridge (1) according to the invention.

### LIST OF REFERENCES

1 Cartridge
10 dimensionally stable outer casing
11 deformation element
12 shell
13 cap
14 base
15 pin
20 collapsible inner tube
21 outlet end
22 outlet opening
23 closed end
24 welded seam
25 broadside of welded seam
26 pouch portion
27 collar portion
30 Inhalation device
X Main axis
L traversal line

1. A cartridge (1) for an inhalation device for the generation and administration of a medically active liquid in aerosolized form, the cartridge comprising
a dimensionally stable outer casing (10),
a deformation element (11), and
a collapsible inner tube (20) for holding the medically active liquid comprising:
an outlet end (21) having an outlet opening (22) and
a closed end (23) opposite of the outlet end, wherein the outlet opening and the (the center of the) closed end defining a main axis (X) of the inner tube connecting the outlet opening and closed end of the tube;
wherein the closed end of the tube is closed by a welded seam (24) oriented transverse (preferably substantially perpendicular) to the main axis of the inner tube;
wherein in the assembled state of the cartridge the collapsible inner tube is located inside the outer casing;
characterized in that
in the assembled state the deformation element (11) contacts the welded seam such that the welded seam is deformed to deviate from a traversal line (L) to the main axis of the inner tube by the contact between the welded seam and the deformation element.

2. The cartridge according to claim 1, wherein the outer casing comprises
a shell (12) comprising an outlet opening and an opposite base opening;
a cap (13) adapted to be attached to the outlet opening of the shell; and
a base (14) adapted to be attached to the base opening of the shell.

3. The cartridge according to claim 2, wherein the deformation element is provided on the base of the outer casing.

4. The cartridge according to claim 2 or 3, wherein the deformation element is provided in the form of at least one pin (15).

5. The cartridge according to claim 3, wherein the deformation element is provided in the form of groove provided on the base of the outer casing.

6. The cartridge according to any one of the preceding claims, wherein the deformation element contacts the welded seam of the collapsible tube only and does not substantially contact the collapsible sidewalls of the collapsible tube.

7. The cartridge according to any one of the preceding claims, wherein the deformation element upon contact with the welded seam of the collapsible tube does not substantially reduce the inner volume of the collapsible tube.

8. The cartridge according to any one of the preceding claims, wherein the collapsible tube comprises a pouch portion (26) and a collar portion (27), wherein the collar portion comprises the outlet opening and is adapted to be attached to the shell of the outer casing of the cartridge.

9. The cartridge according to any one of the preceding claims, wherein the outer casing has a volume in the range about 0.1 to about 200 mL, optionally about 0.5 to about 50 ml, further optionally between about 1 mL to 10 ml.

10. The cartridge according to any one of the preceding claims, wherein the inner tube has a volume in the range of from about 2 to about 8 mL.

11. The cartridge according to any one of the preceding claims, wherein the welded seam is in a radially curved form comprising a deviation of up to 0.5 mm, 1 mm, 1.5 mm, 2 mm, 2.5 mm, 3 mm, 3.5 mm, 4 mm, 4.5 mm, or 5 mm from an ideal straight line at the furthest point.

12. The cartridge according to any one of the preceding claims, wherein the medically active liquid comprises at least one pharmaceutically active ingredient and a liquid vehicle.

13. The cartridge according to claim 12, wherein the liquid vehicle comprises at least one solvent selected from the group consisting of water, ethanol, polyethylene glycol, liquid fatty acids, oils, in particular natural oils, and semifluorinated alkanes.

14. An inhalation device adapted to comprise a cartridge according to any of the preceding claims comprising a cartridge according to any one of the preceding claims.

15. A method for the assembly of a cartridge according to any one of claims 1 to 13, the method comprising the steps of
a) providing an inner tube and a dimensionally stable outer casing comprising a deformation element;
b) introducing the inner tube into outer casing, thereby contacting the welded seam of the inner tube with the deformation element of the outer casing and deforming the welded seam of the inner tube;
c) filling of the inner tube with the medically active liquid;
d) optionally sealing the container after filling.
